(19) United States
(12) Patent Application Publication
(10) Pub. No.: US 2026/0220722 A1
(43) Pub. Date: Jul. 30, 2026

(54) OPERATING METHOD AND SYSTEM OF A MEAT DATA MANAGEMENT PLATFORM

(71) Applicant: DEEPLANT, INC., Goyang-si (KR)

(72) Inventor: Chul-beom KIM, Goyangsi (KR)

(21) Appl. No.: 19/575,122

(22) Filed: Mar. 23, 2026

Related U.S. Application Data
(63) Continuation of application No. PCT/KR2024/013018, filed on Aug. 30, 2024.

(30) Foreign Application Priority Data
Sep. 26, 2023 (KR) 10-2023-0128900

(51) Int. Cl. G06Q 50/12

(52) U.S. Cl. CPC G06Q 50/12

(57) ABSTRACT

An embodiment relates to a method for operating a meat data management platform. The method includes receiving, from a user terminal communicatively connected to a server, registration meat information including user information and an image of meat to be registered, measuring at least one of taste, tenderness, and grade of meat included in the image of the meat to be registered, and matching and registering measured meat measurement information with the registration meat information and the user information. An embodiment provides consistent and highly accurate measurement and determination of taste, tenderness, and grade of meat by an artificial intelligence trained on meat images classified according to taste, tenderness, and grade, and manages data regarding grade, taste, and tenderness of meat. In addition, through a preset interface, a function for easily registering meat and viewing meat data may be provided to a user or an administrator.

### CROSS-REFERENCE TO RELATED APPLICATION

This application is a continuation application of PCT Patent Application No. PCT/KR2024/013018 filed on Aug. 30, 2024, which claims priority to Korean Patent Application No. 10-2023-0128900, filed on Sep. 26, 2023, in the Korean Intellectual Property Office, the disclosure of which is incorporated by reference herein in its entirety.

### BACKGROUND

### Technical Field

The present invention relates to an operating method and system of a meat data management platform using an artificial intelligence model, and more particularly, to an operating technology of a platform that registers and manages meat data such as grade, taste, and tenderness of meat.

### Background Art

Globally, meat consumption is continuously increasing. As of 2021, chicken ranks first in global meat consumption, followed by pork and beef.

In 2021, pork consumption is 32.3 kg, and domestic beef consumption is 12.4 kg per capita. Recent consumers can obtain information about meat through various media and, based on such information, intend to purchase better-quality meat. This is because differences in taste and price are significant depending on the grade of meat.

In purchasing meat, the grade and taste of meat are important factors among consideration factors including price and country of origin. Factors determining the grade and taste of meat may include marbling, meat color, juiciness, and texture.

However, the grade and taste of meat are not easy to determine or measure unless by experts, and evaluations of grade and taste may differ among experts. This is because criteria such as marbling, meat color, texture, and palatability, which are major items for evaluating the grade and taste of meat, vary depending on the evaluating expert and differ by country.

As described above, the absence of objective data supporting evaluation results is the primary cause of a decrease in the reliability of evaluations regarding meat taste, tenderness, and grade (tenderness).

Accordingly, there is an increasing need for an operating method and system of a meat data management platform capable of analyzing a single meat image based on data possessed by an artificial intelligence trained on a plurality of meat images classified according to taste, tenderness, and grade, and rapidly and consistently measuring and managing taste, tenderness, and grade.

### SUMMARY

### Problem to be Solved

The present invention is intended to solve the above-described problems of the related art, and relates to an operating method and system of a meat data management platform using an artificial intelligence model that determines the grade of meat, measures taste and tenderness of meat to perform sensory evaluation, and manages data regarding taste, tenderness, and grade of meat.

However, the technical problem to be achieved by the present embodiment is not limited to the above-described technical problem, and other technical problems may exist.

### Means for Solving the Problem

As a technical means for achieving the above-described technical problem, an embodiment according to a first aspect of the present disclosure provides a method for operating a meat data management platform. The method comprises: receiving, from a user terminal communicatively connected to the server, registration meat information including user information and an image of meat to be registered; measuring at least one of taste, tenderness, and grade of meat included in the image of the meat to be registered; and matching and registering measured meat measurement information with the meat-to-be-registered information and the user information.

In addition, an embodiment according to a second aspect of the present disclosure provides a system for operating a meat data management platform. The system stores code that causes the system to receive, from a user terminal communicatively connected to the server, registration meat information including user information and an image of meat to be registered, measure at least one of taste, tenderness, and grade of meat included in the image of the meat to be registered, and match and register measured meat measurement information with the meat-to-be-registered information and the user information.

### Effects of the Invention

The present invention provides consistent and highly accurate measurement and determination of taste, tenderness, and grade of meat by an artificial intelligence trained on meat images classified according to taste, tenderness, and grade, which registers and manages meat data such as grade, taste, and tenderness of meat, and is capable of managing the data.

In addition, through a preset interface, a function for simply and easily registering meat and viewing meat data may be provided to a user or an administrator.

### BRIEF DESCRIPTION OF THE DRAWINGS

FIG. 1 is a diagram illustrating an operating system of a meat data management platform according to an embodiment of the present invention.

FIG. 2 is a diagram illustrating a detailed configuration of the server shown in FIG. 1.

FIG. 3 is a diagram illustrating a main screen of an application according to an embodiment of the present invention.

FIGS. 4-8 are diagrams illustrating a meat registration process according to an embodiment of the present invention.

FIG. 9 is a diagram illustrating viewing of meat data according to an embodiment of the present invention.

FIG. 10 is a diagram illustrating a main screen of an application according to a second embodiment of the present invention.

FIGS. 11-15 are diagrams illustrating a meat registration process according to the second embodiment of the present invention.

FIGS. 16A-23 are diagrams illustrating an additional data input process of meat according to the second embodiment of the present invention.

FIGS. 24 and 25 are diagrams illustrating confirmation of meat data according to the second embodiment of the present invention.

FIGS. 26-29 are diagrams illustrating viewing of meat data according to the second embodiment of the present invention.

FIG. 30 is a diagram illustrating a process of photographing meat according to an embodiment of the present invention.

FIG. 31 is a flowchart illustrating a sequence of a method for operating a meat data management platform according to another embodiment of the present invention.

### DETAILED DESCRIPTION

Hereinafter, the present disclosure will be described in detail with reference to the accompanying drawings. However, the present disclosure may be implemented in various different forms and is not limited to the embodiments described herein. The accompanying drawings are provided only to facilitate understanding of the embodiments disclosed in the present specification, and the technical spirit disclosed in the present specification is not limited by the accompanying drawings. All terms including technical and scientific terms used herein shall be interpreted as having meanings commonly understood by those skilled in the art to which the present disclosure pertains. Terms defined in advance shall be interpreted as additionally having meanings consistent with related technical documents and the presently disclosed content, and shall not be interpreted in an excessively ideal or restrictive sense unless otherwise defined.

In order to clearly describe the present invention in the drawings, portions unrelated to the description are omitted, and the size, shape, and form of each component illustrated in the drawings may be variously modified. Throughout the specification, identical or similar reference numerals are assigned to identical or similar parts.

Throughout the specification, when a part is described as being “connected (coupled, contacted, or joined)” to another part, this includes not only a case in which the part is directly connected (coupled, contacted, or joined) to the other part, but also a case in which the part is indirectly connected (coupled, contacted, or joined) to the other part with another member interposed therebetween. In addition, when a part is described as “including (having or provided with)” a certain component, this means that other components may be further included (having or provided with), rather than excluding other components, unless specifically stated otherwise.

In the present specification, the term “unit” includes a unit implemented by hardware, a unit implemented by software, and a unit implemented by a combination of both. One unit may be implemented by two or more pieces of hardware, and two or more units may be implemented by one piece of hardware. Meanwhile, the term “unit” is not limited to software or hardware, and may be configured to reside in an addressable storage medium or configured to execute one or more processors. Accordingly, by way of example, the term “unit” includes components such as software components, object-oriented software components, class components, and task components, and includes processes, functions, attributes, procedures, subroutines, segments of program code, drivers, firmware, microcode, circuits, data, databases, data structures, tables, arrays, and variables. Functions provided within components and “units” may be combined into a smaller number of components and “units” or further separated into additional components and “units.” In addition, components and “units” may be implemented to execute one or more CPUs within a device or a secure multimedia card.

In the following description, suffixes such as “module” and “unit” used for components are assigned or used interchangeably only for convenience in drafting the specification, and do not by themselves have mutually distinct meanings or roles. In describing the embodiments disclosed in the present specification, when it is determined that detailed descriptions of related known technologies may obscure the gist of the embodiments disclosed in the present specification, such detailed descriptions are omitted.

Terms indicating ordinal numbers such as first and second used herein are used only for the purpose of distinguishing one component from another component, and do not limit the order or relationship of the components. For example, a first component of the present disclosure may be referred to as a second component, and similarly, a second component may be referred to as a first component. Singular expressions used herein shall be construed as including plural expressions unless the context clearly indicates otherwise.

Hereinafter, a “user terminal” may be implemented as a computer or a portable terminal capable of accessing a server or another terminal through a network. The computer may include, for example, a notebook, a desktop, a laptop, or a VR HMD (for example, HTC VIVE, Oculus Rift, GearVR, DayDream, PSVR, etc.) equipped with a web browser. The VR HMD includes PC-based devices (for example, HTC VIVE, Oculus Rift, FOVE, Deepon, etc.), mobile-based devices (for example, GearVR, DayDream, Baofeng Mojing, Google Cardboard, etc.), console-based devices (PSVR), and stand-alone models independently implemented (for example, Deepon, PICO, etc.). The portable terminal may include, for example, a wireless communication device having portability and mobility, such as a smartphone, a tablet PC, a wearable device, as well as various devices equipped with communication modules such as BLE (Bluetooth Low Energy), NFC, RFID, ultrasonic communication, infrared communication, WiFi, and LiFi. In addition, the “network” refers to a connection structure capable of exchanging information between respective nodes such as terminals and servers, and includes a LAN (Local Area Network), a WAN (Wide Area Network), the Internet (WWW: World Wide Web), wired or wireless data communication networks, telephone networks, and wired or wireless television communication networks. Examples of wireless data communication networks include 3G, 4G, 5G, 3GPP (3rd Generation Partnership Project), LTE (Long Term Evolution), WIMAX (World Interoperability for Microwave Access), Wi-Fi, Bluetooth communication, infrared communication, ultrasonic communication, VLC (Visible Light Communication), and LiFi, but are not limited thereto.

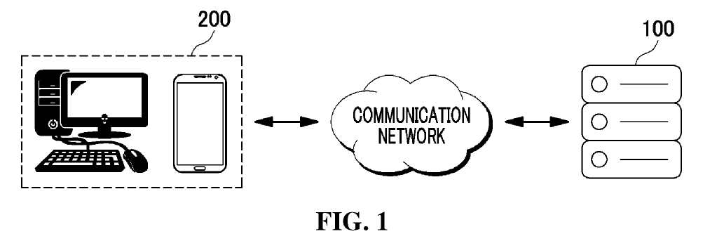

FIG. 1 is a diagram illustrating an operating system of a meat data management platform according to an embodiment of the present invention.

Referring to FIG. 1, the operating system of the meat data management platform includes a server (100) and a user terminal (200), and the server (100) and the user terminal (200) may be communicatively connected through a communication network.

The server (100) may be formed as a cloud computing server such as SaaS (Software as a Service), PaaS (Platform as a Service), or IaaS (Infrastructure as a Service). The server may also be constructed in the form of a private cloud, a public cloud, or a hybrid cloud system, but the scope of the present invention is not limited thereto. In addition, a meat taste and tenderness measurement apparatus (100) may be driven using an artificial intelligence model.

The server receives, from a communicatively connected user terminal, registration meat information including user information and an image of meat to be registered.

The server measures at least one of taste, tenderness, and grade of meat included in the image of the meat to be registered.

The server matches and registers measured meat measurement information with the meat-to-be-registered information and the user information.

The user terminal may transmit, to the server, registration meat information including an image of meat to be registered and user information. The user terminal may receive, from the server, data regarding taste, tenderness, and grade of meat in the form of visualized materials and view the data.

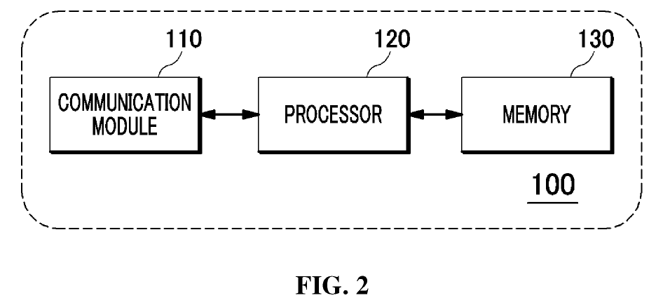

FIG. 2 is a diagram illustrating a detailed configuration of the server shown in FIG. 1.

Referring to FIG. 2, the server (100) may include a communication module (110), a processor (120), and a memory (130).

The communication module (110) may include hardware and software necessary for transmitting and receiving signals such as control signals or data signals through wired or wireless connections with other network devices.

The communication module (110) may receive a registration meat image and user information from the user terminal (200). In addition, the communication module (110) may provide, to the user terminal (200), data regarding taste, tenderness, and grade of meat in the form of visualized materials. However, the present invention is not limited thereto, and the communication module (110) may receive an image or a video from an external device or database other than the user terminal (200), as necessary.

The processor (120) may include various types of devices that control and process data. The processor (120) may refer to a hardware-embedded data processing device having physically structured circuits for performing functions expressed as code or instructions included in a program.

In one example, the processor (120) may be implemented in the form of a microprocessor, a central processing unit (CPU), a processor core, a multiprocessor, an application-specific integrated circuit (ASIC), or a field programmable gate array (FPGA), but the scope of the present invention is not limited thereto.

The processor (120) performs operations according to code stored in the memory (130).

The memory (130) may store at least one of information and data input through the communication module (110), information and data required for functions performed by the processor (120), and data generated according to execution of the processor (120).

The memory (130) shall be construed as collectively referring to a non-volatile storage device that retains stored information even when power is not supplied and a volatile storage device that requires power to retain stored information. The memory (130) may include a magnetic storage media or a flash storage media in addition to a volatile storage device requiring power to retain stored information, but the scope of the present invention is not limited thereto.

The memory (130) is electrically connected to the processor (120), and at least one code executed by the processor (120) is stored therein. The memory (130) stores code that, when executed by the processor (120), causes the processor (120) to perform the following functions and procedures.

The memory stores code that causes the server to receive, from a user terminal communicatively connected to the server, registration meat information including user information and an image of meat to be registered. For example, the user information includes a name of an owner of the user terminal, a company name, an address, an email address, and an identification, and the user information may be stored in a user information table.

The registration meat information may include a request for registering meat by receiving, from the user terminal, breed, part, and cross-sectional image of meat. In addition, the registration meat information may further include a meat basic table, a fresh meat and processed meat sensory inspection table, a heated meat sensory evaluation table, an electronic tongue experiment table, and a deep aging history table. The meat basic table includes a meat management number, a meat-generating user, gender of the meat, grade, year of birth, farm, farm manager, and individual identification number. The fresh meat and processed meat sensory inspection table includes marbling, meat color, texture, and juiciness. The heated meat sensory evaluation table includes flavor, moisture, tenderness, umami, and palatability. The electronic tongue experiment table includes collagen, sourness, bitterness, umami, and richness. The deep aging history table may include date and time.

The memory stores code that causes measurement of at least one of taste, tenderness, period, and grade of meat included in the image of the meat to be registered. For example, taste, tenderness, period, and grade of meat may be measured using an artificial intelligence model trained to measure at least one of grade, taste, period, and tenderness of meat included in an input meat image. In this case, measurement may be provided based on analysis of the meat through one of an RGB camera, a spectral camera, or a hyperspectral camera.

The memory stores code that causes measurement of at least one of taste, tenderness, period, and grade of the meat based on the registration meat information. For example, the period includes a consumption period, a processing period, and a distribution period of the meat, and the consumption period, the processing period, and the distribution period may be measured based on a second time point at which a period of the meat is measured from a first time point at which the meat is initially registered.

The memory stores code that causes matching of measured meat measurement information with the registration meat information and the user information.

The memory stores code that causes assignment of a unique management number to each meat and, when registration of the meat is completed, generation and provision of a QR code containing completed information.

The memory stores code that causes reception of a request for viewing data regarding any one meat from the user terminal and provision of data corresponding to the meat. For example, the data is evidence data including color distribution, and the evidence data may visualize and provide grade, taste, and tenderness. In addition, the data may further include a QR code, deep aging data, cross-sectional photographing data, sensory evaluation data, taste data, and experiment data.

The memory stores code that, when there is an error in the data, causes reception of a data correction request from the user terminal and updating of the data by reflecting correction items input through the user terminal.

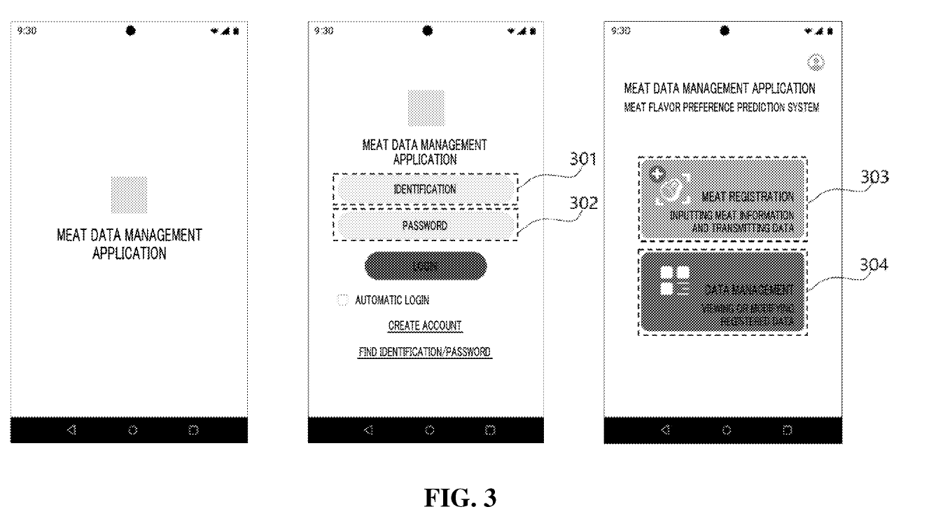

FIG. 3 is a diagram illustrating a main screen of an application according to an embodiment of the present invention.

Referring to FIG. 3, login may be performed by inputting an identification (301) and a password (302) on the main screen of the application. When login is completed, meat registration (303) or data management (304) may be selected and performed. When the application is used for the first time, membership registration is performed before login, and the membership registration may be performed by inputting user information including name, company name, address, email address, and identification.

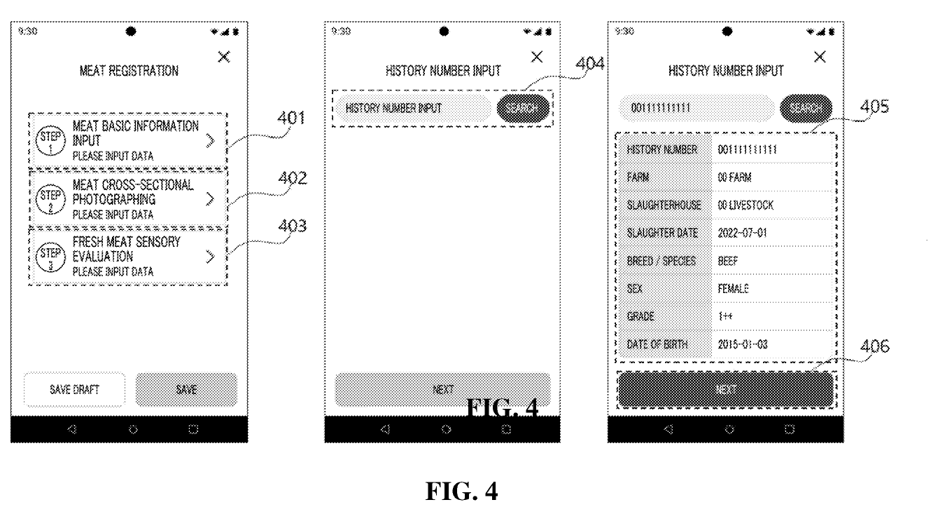

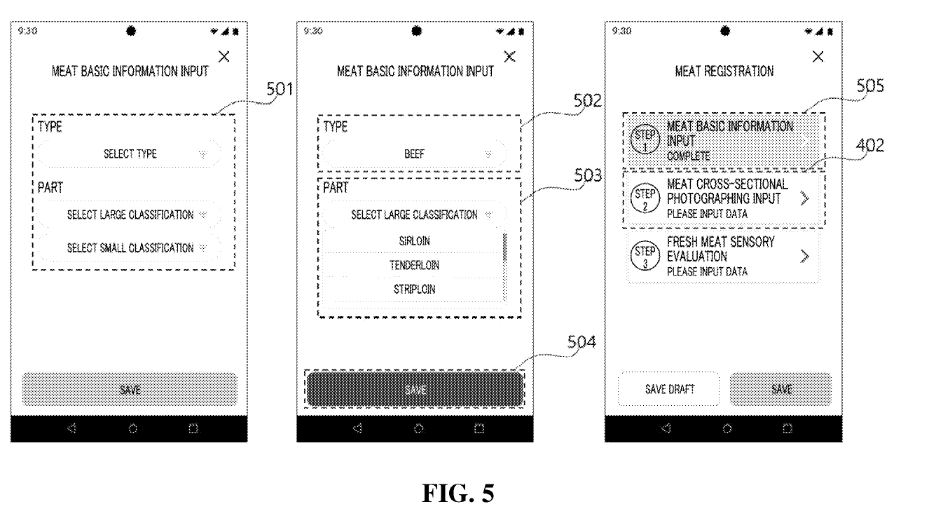

FIGS. 4-8 are diagrams illustrating a meat registration process according to an embodiment of the present invention.

Referring to FIG. 4, for meat registration, meat basic information input (401), meat cross-sectional photographing (402), and fresh meat sensory evaluation (403) may be performed. For meat registration, a history number input (404) is performed, and meat basic information input may be performed by pressing a next button (406) on a search result provision screen (405).

Referring to FIG. 5, for meat basic information input (401), a type and part of meat (501) may be selected. As the type of meat (502), cattle may be selected, and as the part of meat (503), a detailed part may be selected by dividing into a large classification and a small classification. When all selections are completed, a save button (504) is pressed to complete meat basic information input (505), and meat cross-sectional photographing input (402) may be performed.

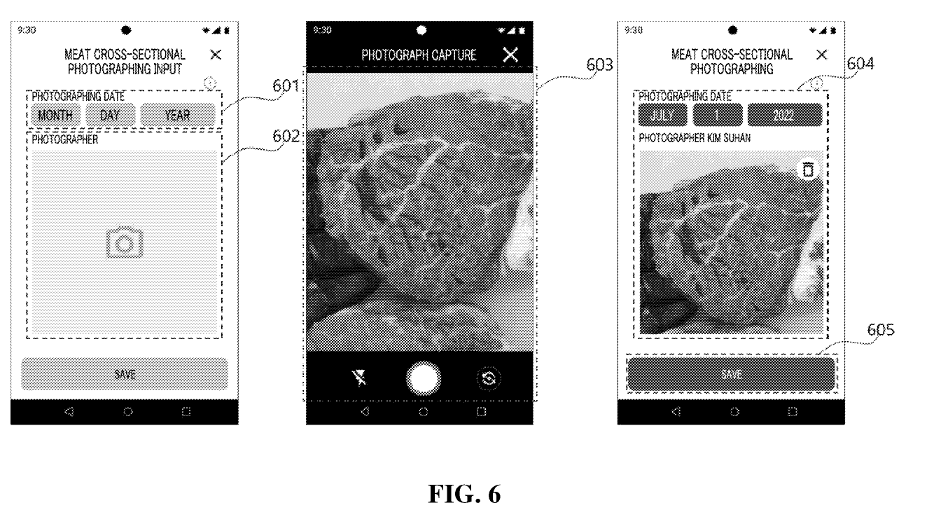

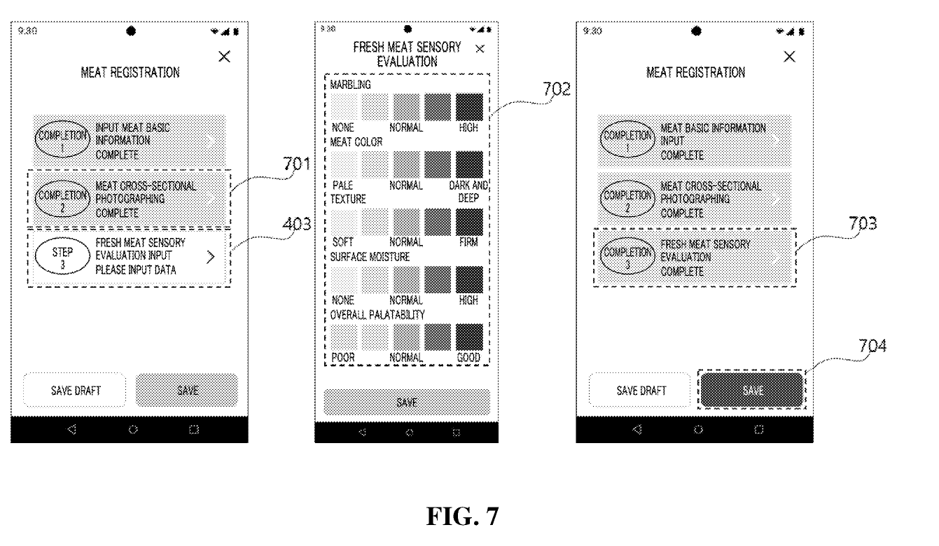

Referring to FIG. 6, for meat cross-sectional photographing input (402), a photographing date (601) may be selected, and a photographer and a photograph may be input (602). When a photograph is taken (603) using a camera of the user terminal, the input photographing date, photographer, and photograph may be provided as a result (604). When there is no abnormality in the result (604), a save button (605) is pressed to complete meat cross-sectional photographing (701).

Referring to FIG. 7, when meat cross-sectional photographing is completed (701), fresh meat sensory evaluation input (403) may be performed. The fresh meat sensory evaluation may be completed by selecting (702) marbling, meat color, texture, surface juiciness, and overall palatability. When the fresh meat sensory evaluation is completed (703), a save button (704) is pressed to complete meat registration.

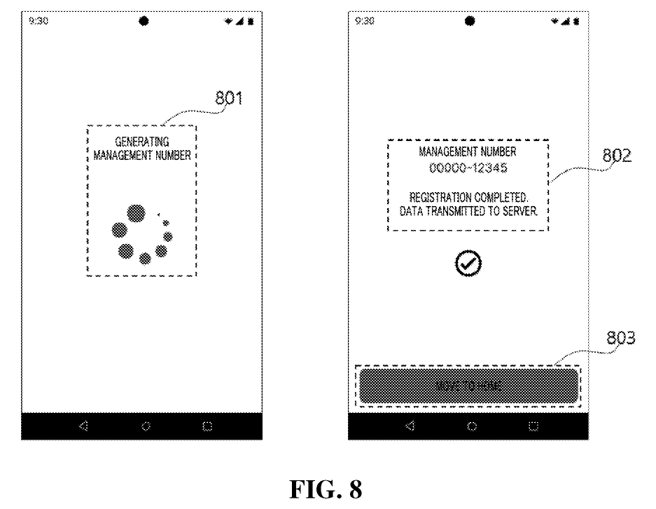

Referring to FIG. 8, a screen in which a management number is being generated (801) after completion of meat registration and a screen in which generation of the management number is completed (802) may be confirmed. After all processes are completed, a move-to-home button (803) is pressed to move to the main screen.

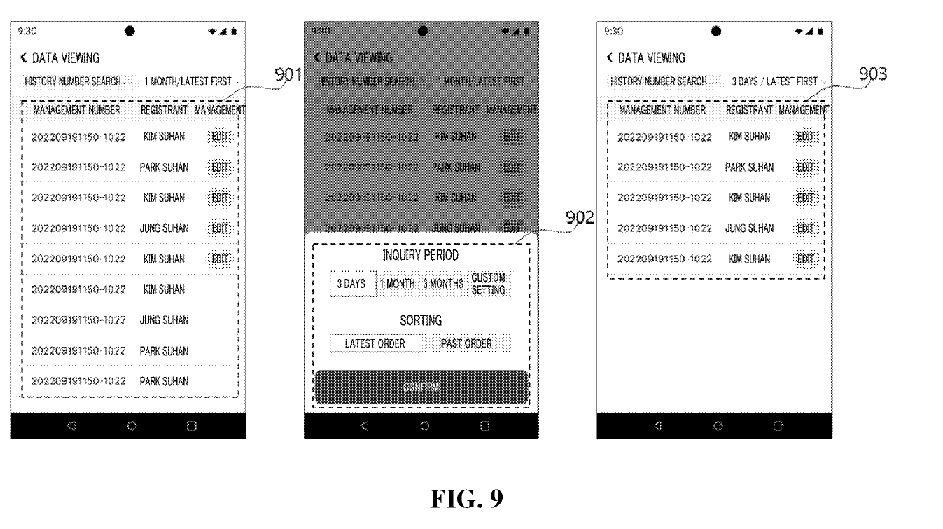

FIG. 9 is a diagram illustrating viewing of meat data according to an embodiment of the present invention.

Referring to FIG. 9, a data viewing screen may be provided (901) including a management number, a registrant, and management items. Filtering and sorting (902) may be performed based on an inquiry period or in latest order or past order. Data corresponding to a selected filter may be provided (903).

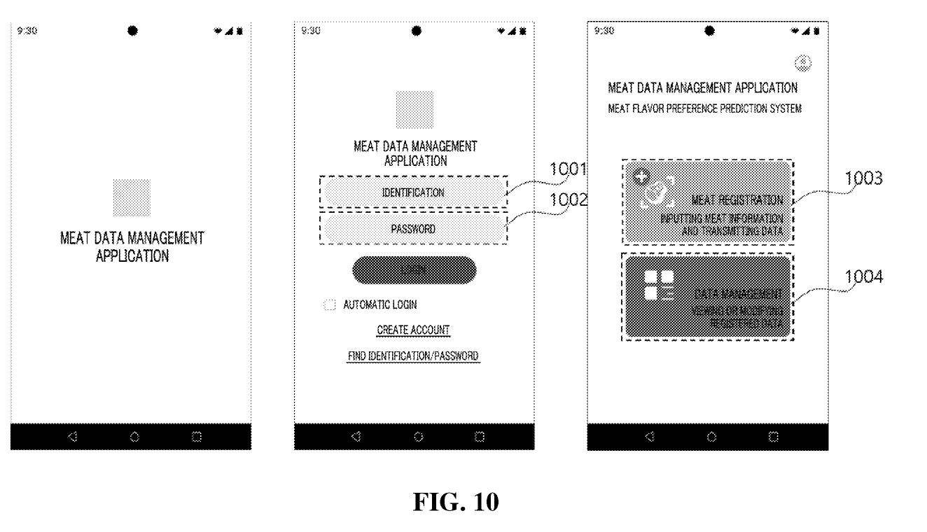

FIG. 10 is a diagram illustrating a main screen of an application according to a second embodiment of the present invention.

Referring to FIG. 10, login may be performed by inputting an identification (1001) and a password (1002) on the main screen of the application. When login is completed, meat registration (1003) or data management (1004) may be selected and performed.

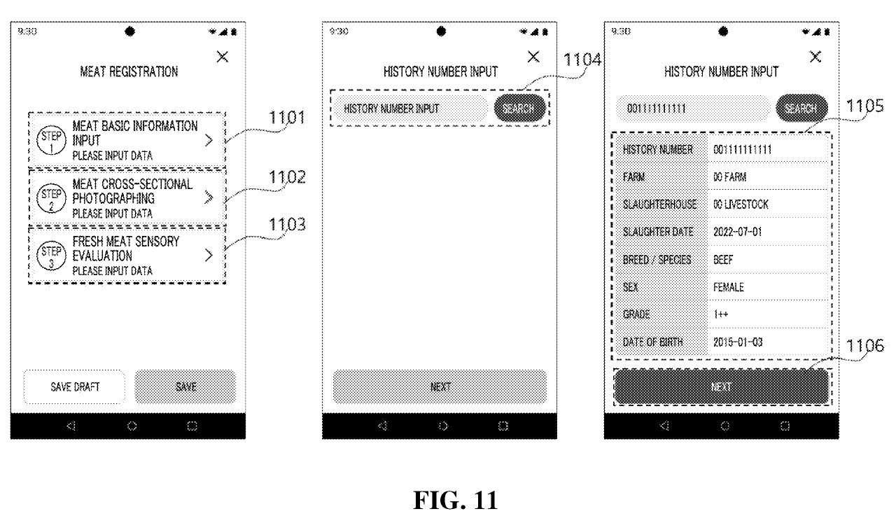

FIGS. 11-15 are diagrams illustrating a meat registration process according to the second embodiment of the present invention.

Referring to FIG. 11, for meat registration, meat basic information input (1101), meat cross-sectional photographing (1102), and fresh meat sensory evaluation (1103) may be performed. For meat registration, a history number input (1104) is performed, and meat basic information input may be performed by pressing a next button (1106) on a search result provision screen (1105).

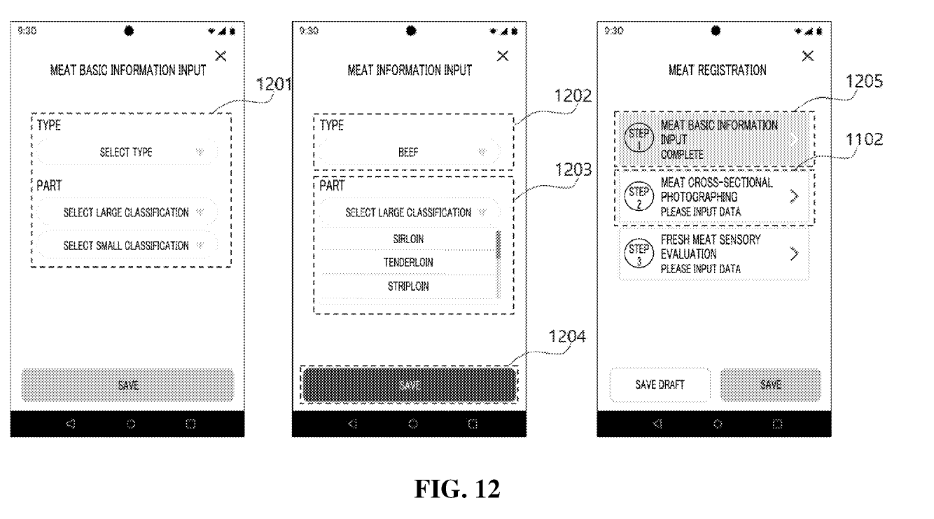

Referring to FIG. 12, for meat basic information input (1101), a type and part of meat (1201) may be selected. As the type of meat (1202), cattle may be selected, and as the part of meat (1203), a detailed part may be selected by dividing into a large classification and a small classification. When all selections are completed, a save button (1204) is pressed to complete meat basic information input (1205), and meat cross-sectional photographing (1206) may be performed.

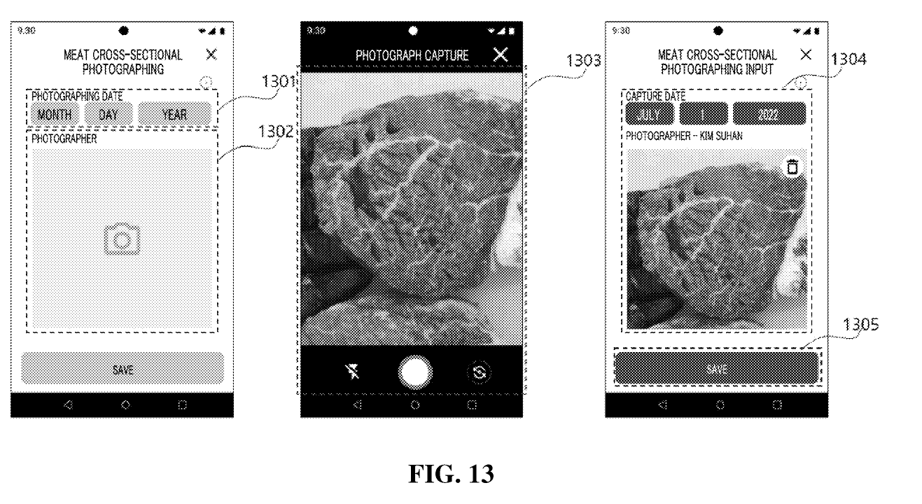

Referring to FIG. 13, for meat cross-sectional photographing input (1102), a photographing date (1301) may be selected, and a photographer and a photograph may be input (1302). When a photograph is taken (1303) using a camera of the user terminal, the input photographing date, photographer, and photograph may be provided as a result (1304). When there is no abnormality in the result (1304), a save button (1305) is pressed to complete meat cross-sectional photographing (1401).

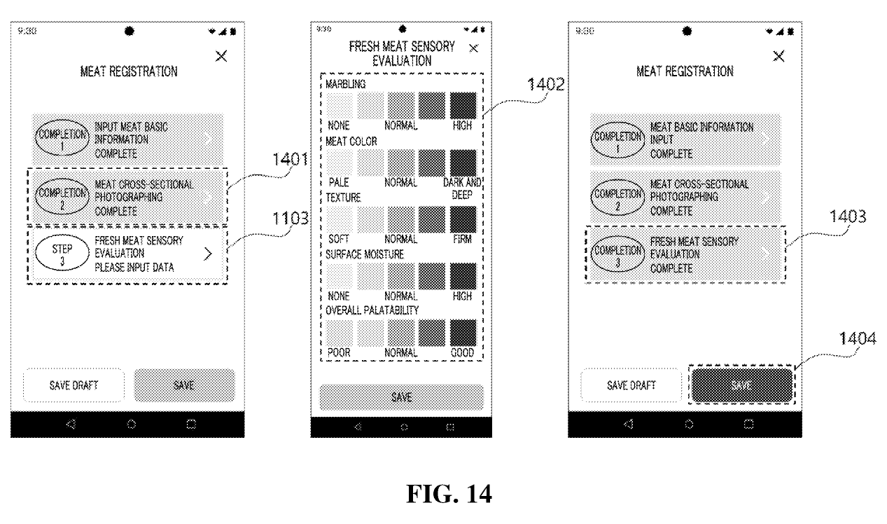

Referring to FIG. 14, when meat cross-sectional photographing is completed (1401), fresh meat sensory evaluation input (1103) may be performed. The fresh meat sensory evaluation may be completed by selecting (1402) marbling, meat color, texture, surface juiciness, and overall palatability. When the fresh meat sensory evaluation is completed (1403), a save button (1404) is pressed to complete meat registration.

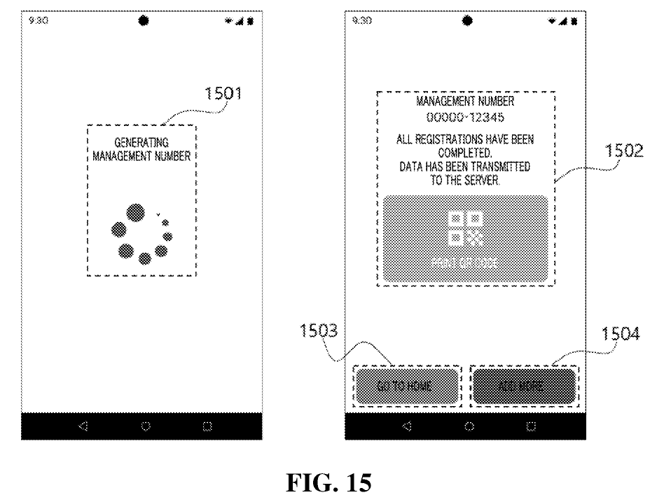

Referring to FIG. 15, a screen in which a management number is being generated (1501) after completion of meat registration and a screen in which generation of the management number is completed (1502) may be confirmed. After all processes are completed, a move-to-home button (1503) or an additional information input button (1504) may be pressed to perform a desired operation.

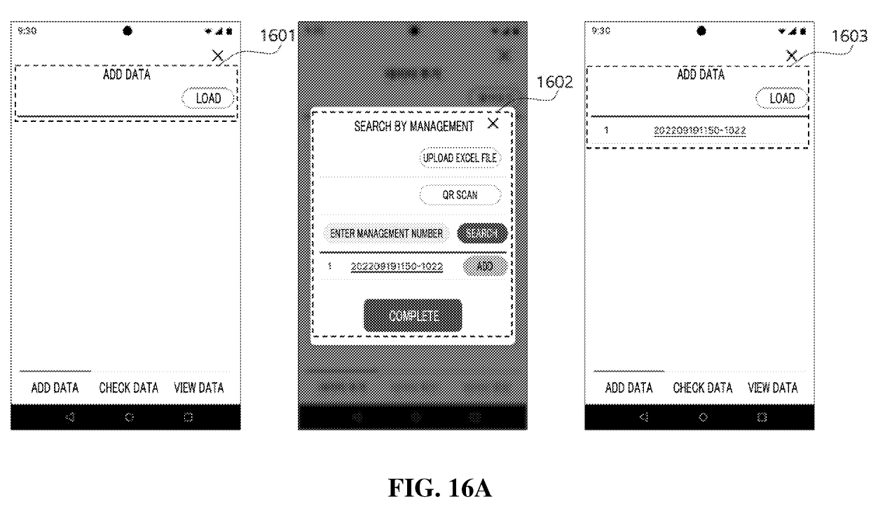

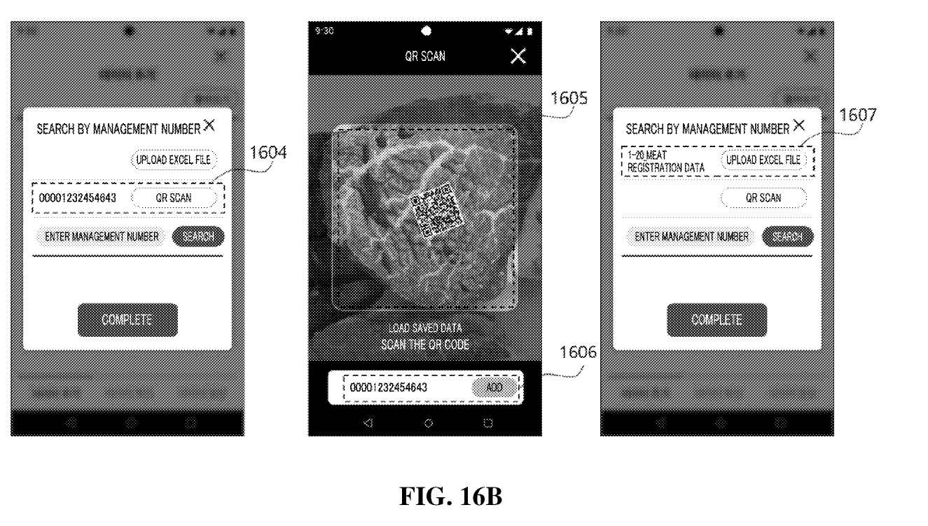

FIGS. 16A-23 are diagrams illustrating an additional data input process of meat according to the second embodiment of the present invention.

Referring to FIG. 16A, when the additional information input button (1504) is pressed in FIG. 15, a screen provided for data addition includes a load button (1601). In a management number load pop-up, additional information may be input to searched data (1603) through Excel file upload, QR scanning, or management number input (1602). For QR code scanning, when a QR scan button (1604) is pressed as shown in FIG. 16B, a preset screen (1605) is provided, and when the QR code is visible to a camera, a corresponding code may be automatically input (1606). Alternatively, an Excel file upload button (1607) may be pressed to register a file.

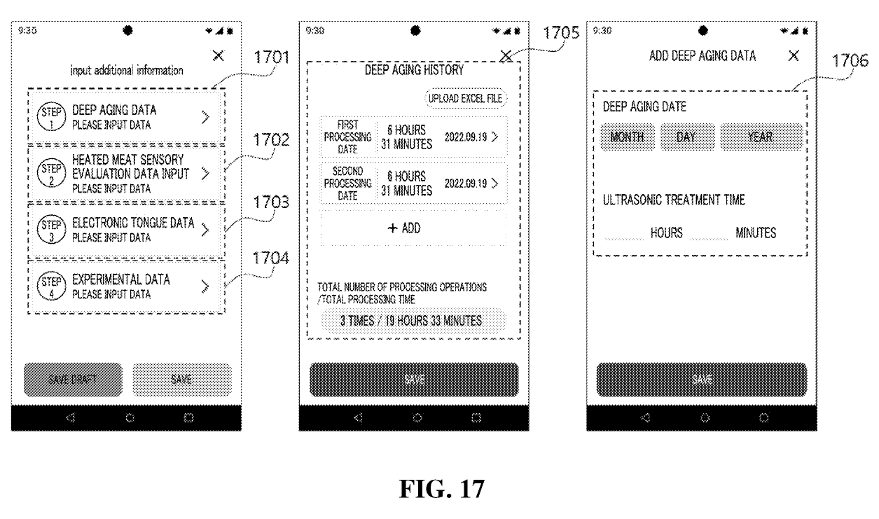

Referring to FIG. 17, for additional information input, deep aging data input (1701), heated meat sensory evaluation data input (1702), electronic tongue data input (1703), and experiment data input (1704) may be performed. In deep aging data input (1701), order, time, and date are displayed (1705), and when adding deep aging data, a deep aging date including month, day, and year and an ultrasonic processing time may be input (1706).

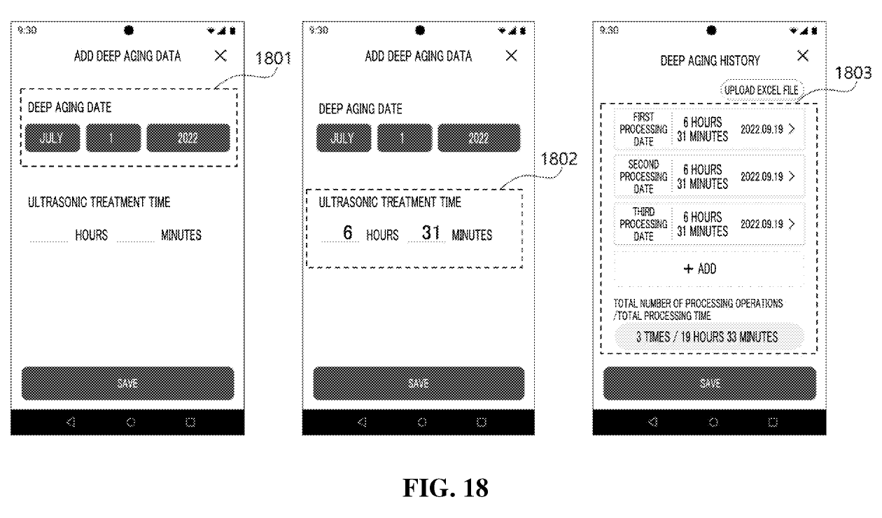

Referring to FIG. 18, when a deep aging date is input as Jul. 1, 2022 (1801), and an ultrasonic processing time is input as 6 hours and 31 minutes (1802) and saved, a total number of first, second, and third deep aging data entries (three times) and a total required time (19 hours and 33 minutes) may be confirmed (1803).

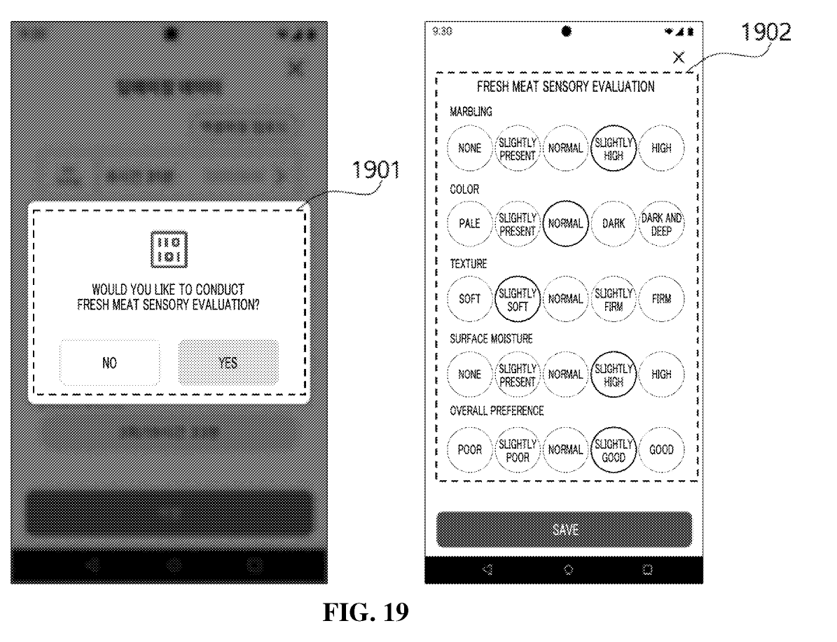

Referring to FIG. 19, a screen inquiring whether to perform fresh meat sensory evaluation is provided as a pop-up (1901), and fresh meat sensory evaluation items (1902) including marbling, meat color, texture, surface juiciness, and overall palatability may be provided.

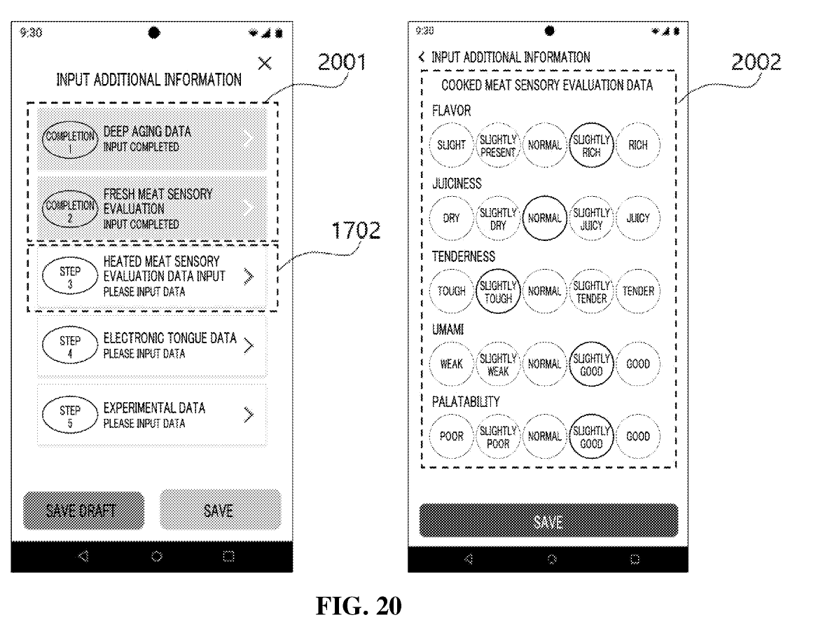

Referring to FIG. 20, when deep aging data and fresh meat sensory evaluation are completed (2001), heated meat sensory evaluation data input (1702) may be performed. The heated meat sensory evaluation data may include flavor, juiciness, tenderness, umami, and palatability (2002).

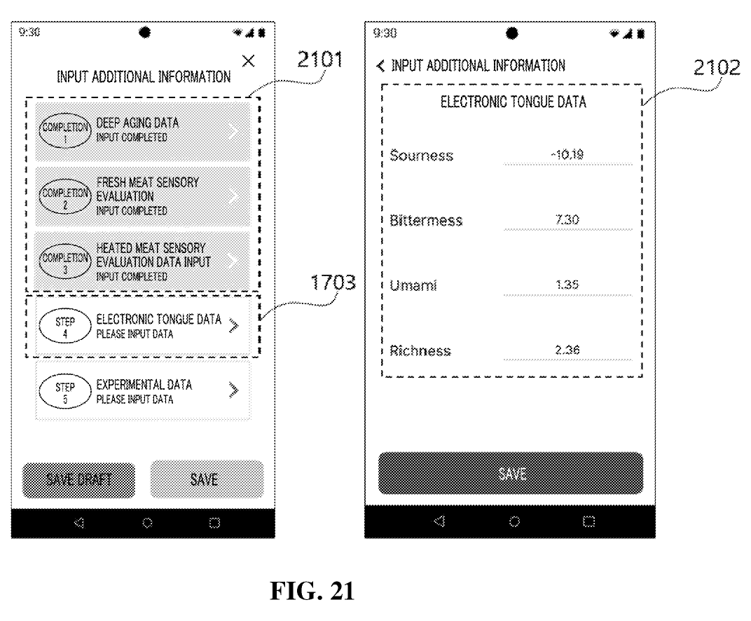

Referring to FIG. 21, when deep aging data, fresh meat sensory evaluation, and heated meat sensory evaluation are completed (2101), electronic tongue data input (1703) may be performed. The electronic tongue data may include sourness, richness, umami, and aftertaste (2102).

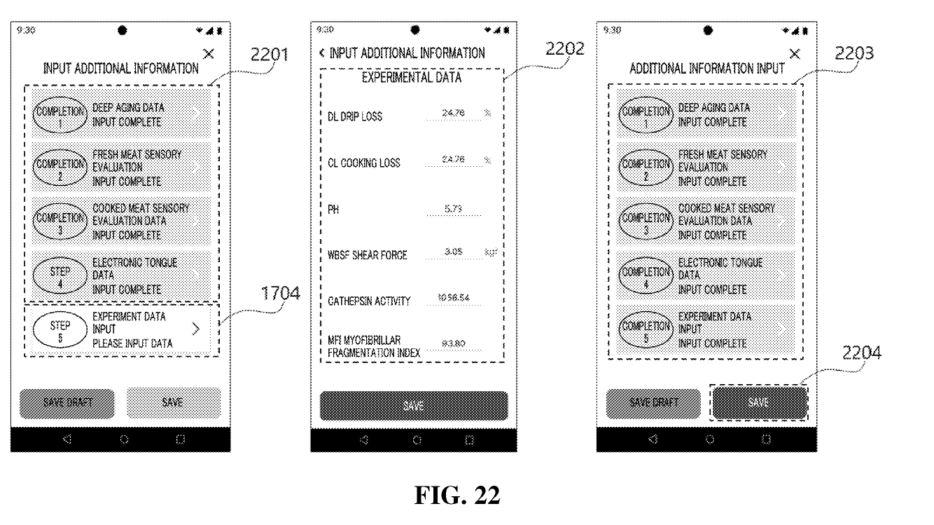

Referring to FIG. 22, when deep aging data, fresh meat sensory evaluation, heated meat sensory evaluation, and electronic tongue data are completed (2201), experiment data input (1704) may be performed. The experiment data may include DL drip loss, CL cooking loss, pH, WBSF shear force, calpain activity, and MFI myofibril fragmentation index (2202). When input of the experiment data is completed, a screen indicating completion of deep aging data, fresh meat sensory evaluation, heated meat sensory evaluation, electronic tongue data, and experiment data (2204) may be provided.

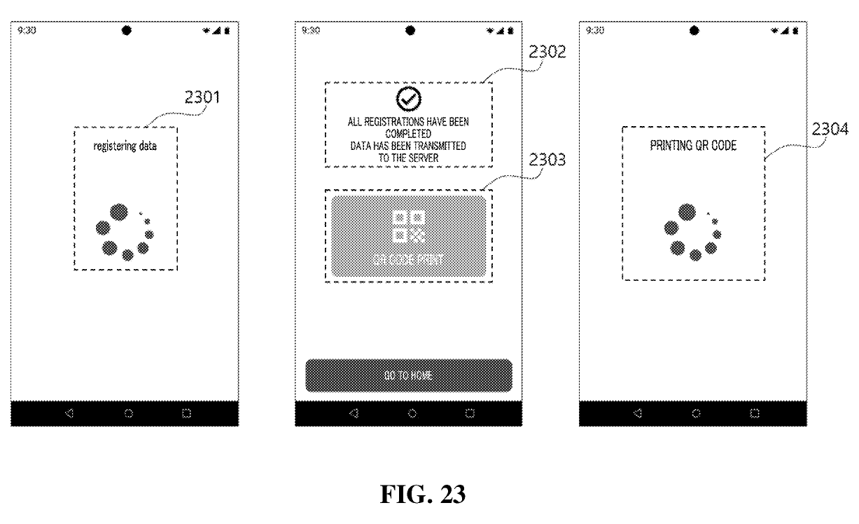

Referring to FIG. 23, a screen indicating that additional data are being registered (2301) after completion of additional data input and a screen indicating that additional data registration is completed (2302) and displaying a QR code print button (2303) may be provided. When the QR code print button (2303) is pressed, a screen indicating that the QR code is being printed (2304) may be provided.

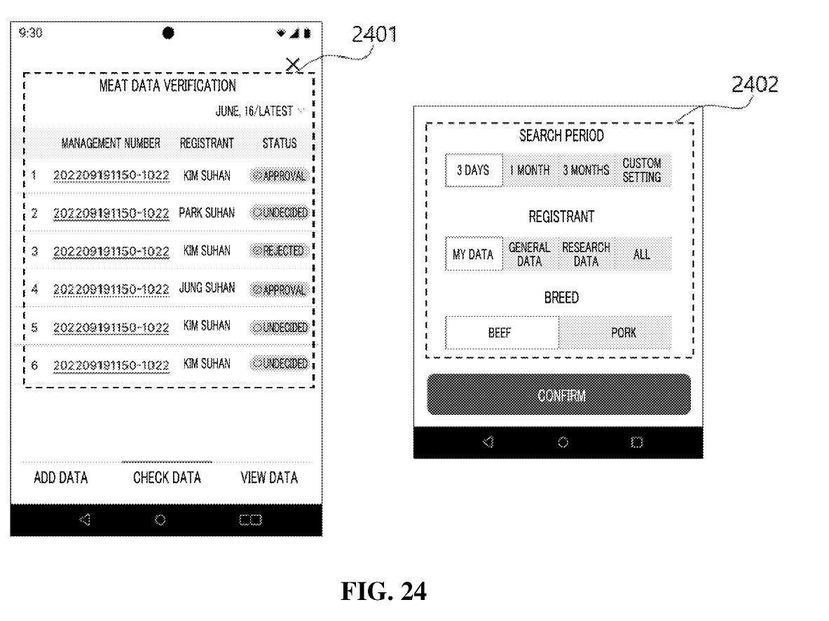

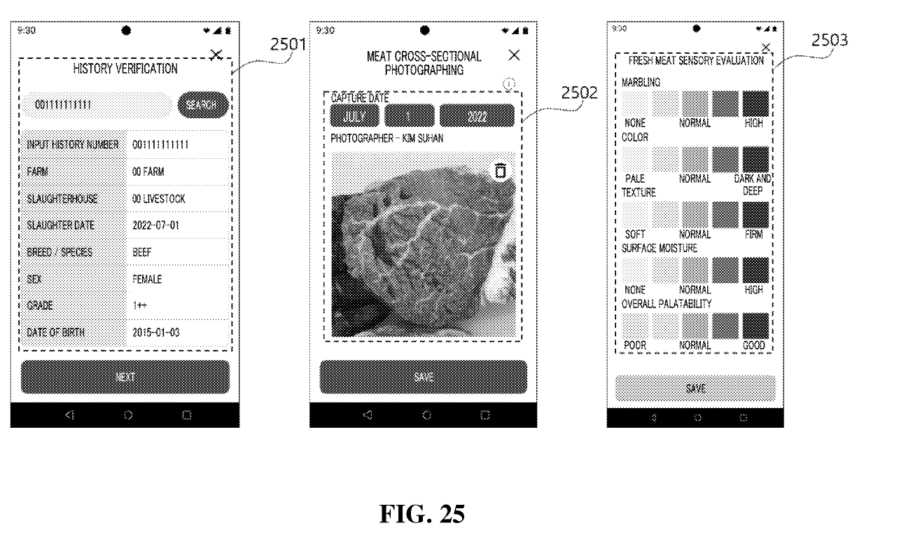

FIGS. 24 and 25 are diagrams illustrating confirmation of meat data according to the second embodiment of the present invention.

Referring to FIG. 24, a meat data confirmation page (2401) including a management number, a registrant, and management items is provided, and meat data may be confirmed through filtering (2402) by inquiry period, registrant, and breed.

Referring to FIG. 25, by inputting a history number, history information (2501) including history number, farm, slaughterhouse, slaughter date, breed or livestock type, gender, grade, and date of birth may be confirmed. In addition, meat cross-sectional data (2502) including photographing date, photographer, and meat image may be confirmed, and fresh meat sensory evaluation data (2503) of the meat may be confirmed.

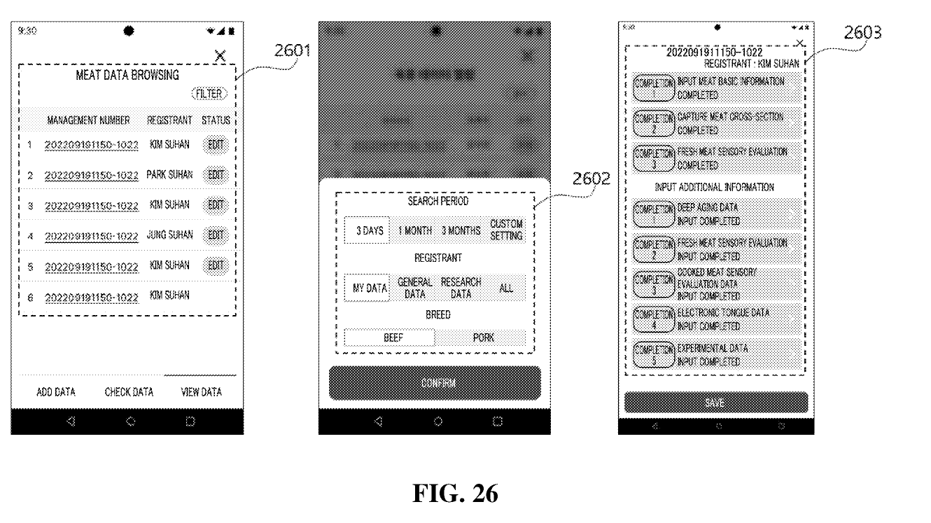

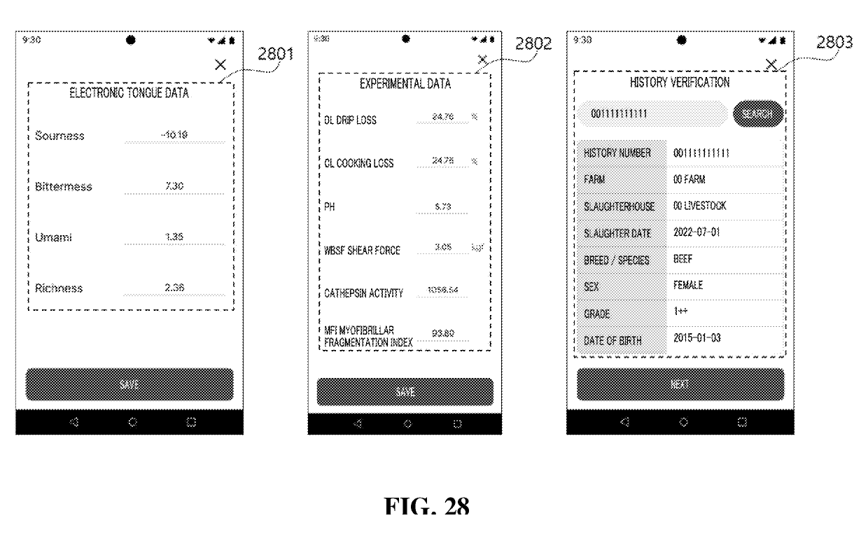

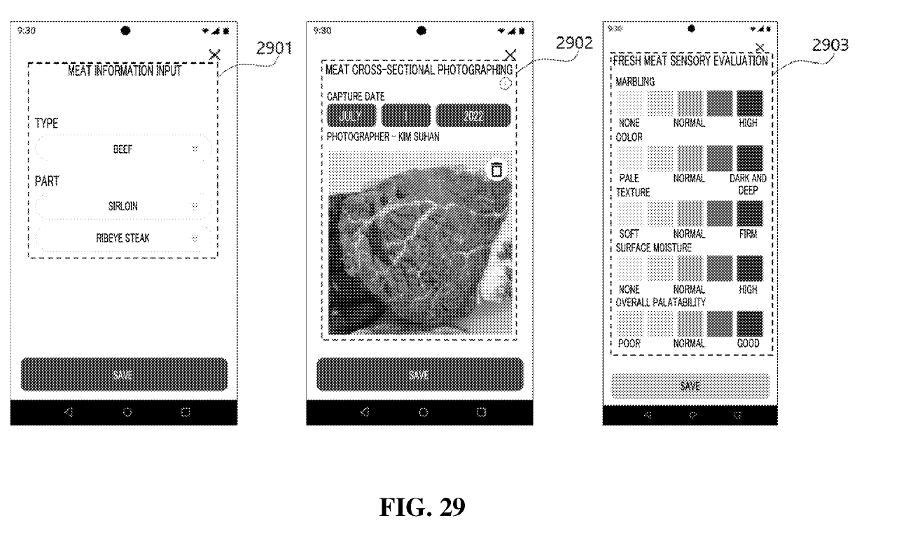

FIGS. 26-29 are diagrams illustrating viewing of meat data according to the second embodiment of the present invention.

Referring to FIG. 26, a meat data viewing page (2601) including a management number, a registrant, and management items is provided, and meat data may be confirmed through filtering (2602) by inquiry period, registrant, and breed. When any one meat data is selected, basic information and additional information including meat basic information, meat cross-section, and fresh meat sensory evaluation may be confirmed (2603).

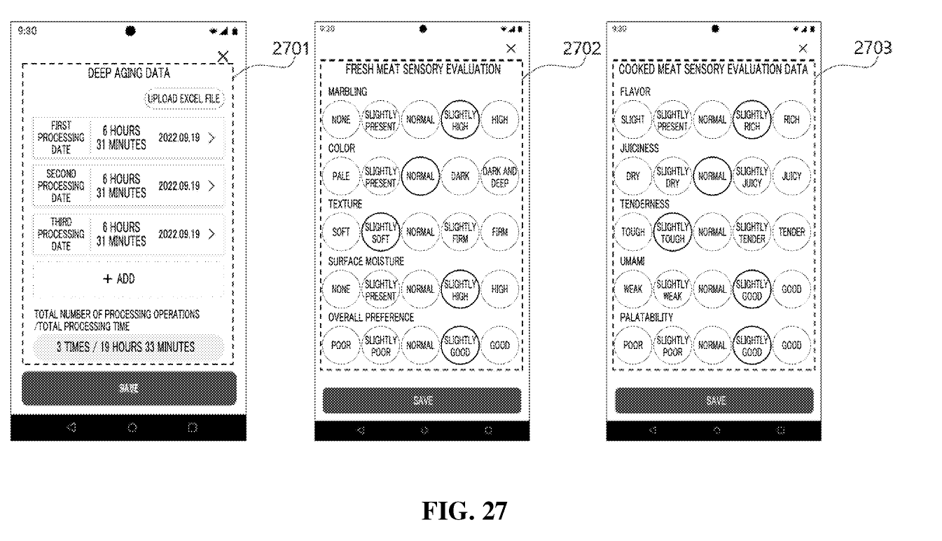

Referring to FIGS. 27-29, deep aging data (2701), fresh meat sensory evaluation (2702), heated meat sensory evaluation data (2703), electronic tongue data (2801), experiment data (2802), meat history (2803), meat information (2901), meat cross-sectional image (2902), and fresh meat sensory evaluation (2903) may be viewed.

As a third embodiment of the present invention, the server may be referred to as a meat grade determination apparatus or a meat taste and tenderness measurement apparatus.

In this case, when a meat image is received from a user terminal, the meat grade determination apparatus or the meat taste and tenderness measurement apparatus may segment and crop a portion of the meat to be evaluated and reset a size of the image. Through this, loss of information may be minimized. Transfer learning may then be performed on the resized image using a pre-trained artificial intelligence model. Thereafter, the grade of the meat may be determined using the artificial intelligence model. Alternatively, the size-adjusted meat image may be latent-vectorized using an artificial intelligence model, and taste and tenderness may be measured based on pre-learned evaluation values for marbling, meat color, texture, juiciness, and palatability by grade, and a sensory evaluation table may be generated.

At this time, the artificial intelligence model used for transfer learning and latent vectorization may use a CNN (Convolutional Neural Network) and a ViT (Vision Transformer).

A CNN is a deep learning model that detects features of an image through convolution operations and, based on the detected features, classifies input data or extracts and learns meaningful information.

A ViT is a deep learning model that uses an attention mechanism for image processing and is effective in extracting global information and performing classification by converting an image into tokens.

More specifically, image tasks are implemented in a Transformer-structured model without using a CNN for vision tasks, and are implemented using a multi-head attention structure of the Transformer. Accordingly, the ViT exhibits excellent performance compared to other CNN-based models and consumes fewer computational resources during a training process.

At this time, larger values are emphasized based on Q (Query), K (Key), and V (Value) values, and these correspond to element representations that are core to learning.

Training management of the artificial intelligence model as described above may use a preset library. The library supports tracking experiments of machine learning models and sharing models.

The meat grade determination apparatus or the meat taste and tenderness measurement apparatus may receive a meat image from a user terminal and present a color distribution graph corresponding to the grade in combination therewith. In this case, the meat image and the color distribution graph may be arranged to overlap each other, or may be arranged side by side. In addition, a sensory evaluation table may be generated using the artificial intelligence model. The sensory evaluation table may include marbling, meat color, texture, surface juiciness, and palatability.

The meat grade determination apparatus or the meat taste and tenderness measurement apparatus may visualize and provide a contribution level for each meat grade using a color distribution graph corresponding to each grade. In this case, the meat image may be presented in combination with the color distribution graph. In addition, the meat may be classified into grades including 1++, 1+, grade 1, grade 2, and grade 3, or taste and tenderness of the meat may be classified. Grad-CAM technology may be applied according to the corresponding meat grade or taste and tenderness to provide a visualized result indicating which portion contributes significantly, wherein a portion closer to red in the meat image indicates a portion contributing more to prediction of the model.

Grad-CAM (Gradient-weighted Class Activation Mapping) is a technique for visualizing prediction results of a deep learning model and may emphasize and display how much a specific portion of an image contributes to a decision of the model. Through this, it is possible to visually understand which portion is mainly utilized by the model and interpret prediction of the model.

The meat grade determination apparatus or the meat taste and tenderness measurement apparatus may generate a latent vector from a meat image received from a user terminal using an artificial intelligence model based on ViT (Vision Transformer), and resize the latent vector to have the same size as an original image for visualization.

As a technique used for visualizing the image, Grad-CAM (Gradient-weighted Class Activation Mapping) and an Attention Map may be used.

For raw meat (fresh meat), taste and tenderness evaluation results may provide marbling, meat color, texture, juiciness, and palatability based on an original image, and may provide marbling, meat color, texture, juiciness, palatability, and an artificial intelligence grade number based on an image visualized by the artificial intelligence model. In addition, a QR code, a management number, a registrant email, and a storage time may be provided together.

For processed meat, taste and tenderness evaluation results may provide marbling, meat color, texture, juiciness, palatability, a sequence number, and a time point based on an original image, and may provide marbling, meat color, texture, juiciness, palatability, a sequence number, a time point, and an artificial intelligence grade number based on an image visualized by the artificial intelligence model. In addition, a QR code, a management number, a registrant email, and a storage time may be provided together.

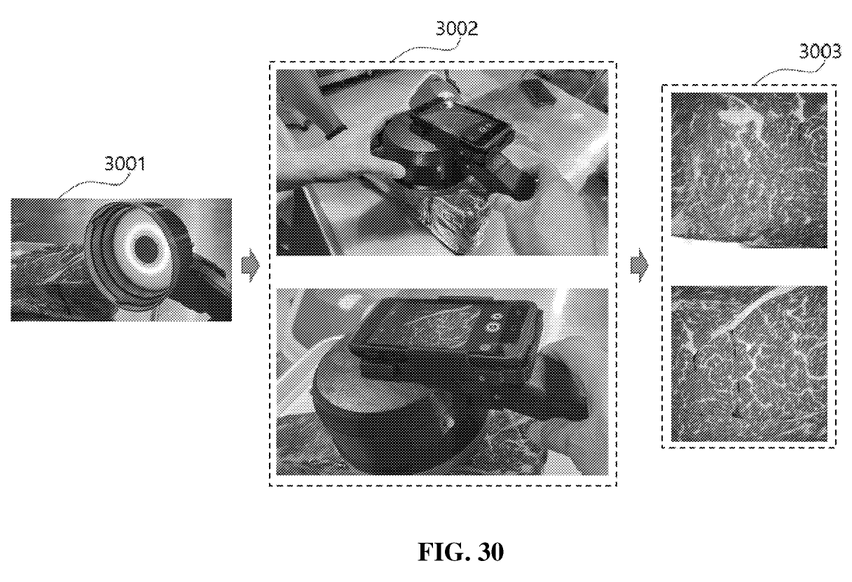

FIG. 30 is a diagram illustrating a process of photographing meat according to an embodiment of the present invention.

Referring to FIG. 30, meat and measurement equipment are prepared (3001), and when a cross-section of the meat is photographed (3002) using the measurement equipment, an image (3003) of the cross-section of the meat may be obtained. In this case, the measurement equipment may be one of a general RGB camera, a spectral camera, or a hyperspectral camera. At least one of taste, tenderness, period, and grade of the meat may be measured from the photographed image of the meat.

In this case, the period may be one of a consumption period, a processing period, or a distribution period, and these periods may be directly related to freshness of the meat according to elapsed days. Accordingly, a sensor capable of detecting a degree of spoilage of the meat may be included in the camera.

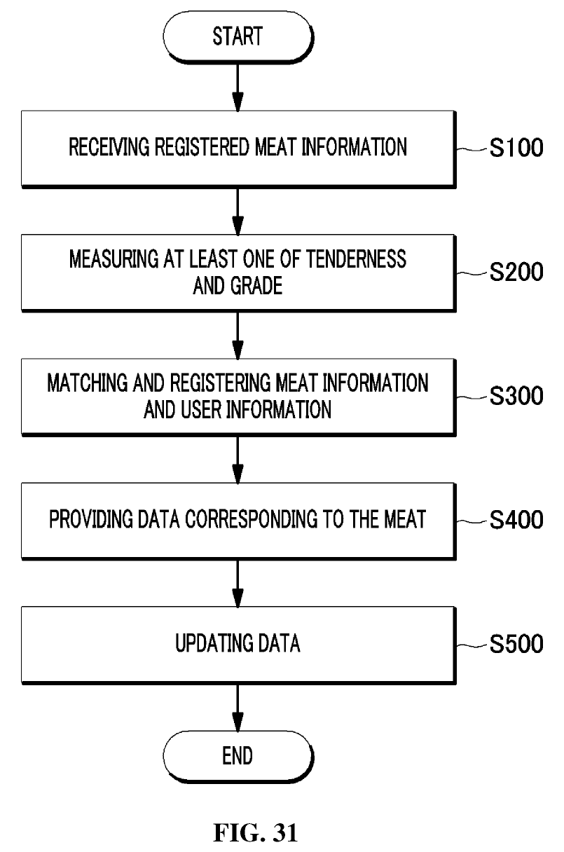

FIG. 31 is a flowchart illustrating a sequence of a method for operating a meat data management platform according to another embodiment of the present invention.

A platform screen door control method described below may be performed by the operating system of the meat data management platform described above with reference to FIGS. 1-30. Accordingly, the embodiments of the present disclosure described above with reference to FIGS. 1-30 may be equally applied to the embodiments described below, and redundant descriptions will be omitted. The steps described below are not necessarily performed in order, the order of the steps may be variously set, and the steps may be performed substantially simultaneously.

Referring to FIG. 31, the method for operating a meat data management platform includes a registration meat information receiving step (S100), a step (S200) of measuring at least one of tenderness and grade, a step (S300) of matching and registering meat information and user information, a step (S400) of providing data corresponding to meat, and a data updating step (S500).

The registration meat information receiving step (S100) is a step of receiving, from a user terminal communicatively connected to the server, registration meat information including user information and an image of meat to be registered. In this case, the user information includes a name of an owner of the user terminal, a company name, an address, an email address, and an identification, and the user information may be stored in a user information table. The registration meat information may include receiving a meat registration request by receiving breed, part, and a cross-sectional image of meat from the user terminal.

In addition, the registration meat information may further include a meat basic table, a fresh meat and processed meat sensory inspection table, a heated meat sensory evaluation table, an electronic tongue experiment table, and a deep aging history table. The meat basic table includes a meat management number, a meat-generating user, gender of the meat, grade, year of birth, farm, farm manager, and individual identification number. The fresh meat and processed meat sensory inspection table includes marbling, meat color, texture, and juiciness. The heated meat sensory evaluation table includes flavor, moisture, tenderness, umami, and palatability. The electronic tongue experiment table includes collagen, sourness, richness, umami, and aftertaste. The deep aging history table may include date and time.

Based on the registration meat information, at least one of taste, tenderness, period, and grade of the meat may be measured.

The step (S200) of measuring at least one of tenderness and grade is a step of measuring at least one of taste, tenderness, period, and grade of meat included in the image of the meat to be registered. In this case, taste, tenderness, period, and grade of the meat may be measured using an artificial intelligence model trained to measure at least one of grade, taste, period, and tenderness of meat included in an input meat image.

The period includes a consumption period, a processing period, and a distribution period of the meat, and the consumption period, the processing period, and the distribution period may be measured based on a second time point at which a period of the meat is measured from a first time point at which the meat is initially registered. The measurement may be provided based on analysis of the meat through one of an RGB camera, a spectral camera, or a hyperspectral camera.

The step (S300) of matching and registering meat information and user information is a step of matching and registering measured meat measurement information with registration meat information and user information. For example, a unique management number may be assigned to each meat, and when registration of the meat is completed, a QR code containing completed information may be generated and provided.

The step (S400) of providing data corresponding to meat is a step of receiving a request for viewing data regarding any one meat from the user terminal and providing data corresponding to the meat. In this case, the data may further include a QR code, deep aging data, cross-sectional photographing data, sensory evaluation data, taste data, and experiment data.

The data updating step (S500) is a step of, when there is an error in the data, receiving a data correction request from the user terminal and updating the data by reflecting correction items input through the user terminal.

An embodiment of the present invention may also be implemented in the form of a recording medium including computer-executable instructions, such as program modules executed by a computer. A computer-readable medium may be any available medium that can be accessed by a computer and includes both volatile and non-volatile media, removable and non-removable media. In addition, the computer-readable medium may include computer storage media. Computer storage media include volatile and non-volatile, removable and non-removable media implemented in any method or technology for storage of information such as computer-readable instructions, data structures, program modules, or other data.

Although the method and system of the present invention have been described in connection with specific embodiments, some or all of components or operations thereof may be implemented using a computer system having a general-purpose hardware architecture.

Those skilled in the art to which the present disclosure pertains will understand that various modifications may be easily made in other specific forms without changing the technical spirit or essential features of the present disclosure based on the foregoing description. Accordingly, the embodiments described above are to be understood as illustrative in all respects and not restrictive. The scope of the present disclosure is defined by the appended claims, and all changes or modifications derived from the meaning and scope of the claims and their equivalents shall be construed as being included within the scope of the present disclosure. The scope of the present application is defined by the appended claims rather than the foregoing detailed description, and all changes or modifications derived from the meaning and scope of the claims and their equivalents shall be construed as being included within the scope of the present application.

What is claimed is:

1. A method for operating a meat data management platform performed by a server, the method comprising:
a) receiving, from a user terminal communicatively connected to the server, registration meat information including user information and an image of meat to be registered;
b) measuring at least one of taste, tenderness, period, and grade of meat included in the image of the meat to be registered; and
c) matching and registering meat measurement information measured according to step b) with the registration meat information and the user information according to step a).

2. The method of claim 1,
wherein, in step b), the taste, tenderness, period, and grade of the meat are measured using an artificial intelligence model trained to measure at least one of grade, taste, period, and tenderness of meat included in an input meat image.

3. The method of claim 1,
wherein the user information includes a name of an owner of the user terminal, a company name, an address, an email address, and an identification, and the user information is stored in a user information table.

4. The method of claim 1,
wherein the registration meat information includes receiving a meat registration request by receiving, from the user terminal, breed, part, and a cross-sectional image of meat.

5. The method of claim 4,
wherein the registration meat information further includes a meat basic table, a fresh meat and processed meat sensory inspection table, a heated meat sensory evaluation table, an electronic tongue experiment table, and a deep aging history table,
wherein the meat basic table includes a meat management number, a meat-generating user, gender of the meat, grade, year of birth, farm, farm manager, and individual identification number,
wherein the fresh meat and processed meat sensory inspection table includes marbling, meat color, texture, and juiciness,
wherein the heated meat sensory evaluation table includes flavor, moisture, tenderness, umami, and palatability,
wherein the electronic tongue experiment table includes collagen, sourness, richness, umami, and aftertaste,
wherein the deep aging history table includes date and time, and
wherein step b) includes measuring at least one of taste, tenderness, period, and grade of the meat based on the registration meat information.

6. The method of claim 1,
wherein step c) further includes assigning a unique management number to each meat, and generating and providing a QR code containing completed information when registration of the meat is completed.

7. The method of claim 1, further comprising:
d) receiving, from the user terminal, a request for viewing data regarding any one meat, and providing data corresponding to the meat,
wherein the data is evidence data including a color distribution, and the evidence data visualizes and provides grade, taste, and tenderness.

8. The method of claim 7,
wherein the data further includes a QR code, deep aging data, cross-sectional photographing data, sensory evaluation data, taste data, and experiment data.

9. The method of claim 1, further comprising:
e) when there is an error in the data in step d), receiving a data correction request from the user terminal and updating the data by reflecting correction items input through the user terminal.

10. The method of claim 2,
wherein the period includes a consumption period, a processing period, and a distribution period of the meat, and
wherein the consumption period, the processing period, and the distribution period are measured based on a second time point at which a period of the meat is measured from a first time point at which the meat is initially registered.

11. The method of claim 1,
wherein the measuring is provided based on analysis of the meat through one of an RGB camera, a spectral camera, or a hyperspectral camera.

12. A system for operating a meat data management platform, comprising:
a communication module;
at least one processor; and
a memory electrically connected to the processor and storing at least one code executed by the processor,
wherein the memory stores code that, when executed by the processor, causes the processor to receive, from a user terminal communicatively connected to the server, registration meat information including user information and an image of meat to be registered, measure at least one of taste, tenderness, period, and grade of meat included in the image of the meat to be registered, and match and register measured meat measurement information with the registration meat information and the user information.

13. The system of claim 12,
wherein the taste, tenderness, period, and grade of the meat are measured using an artificial intelligence model trained to measure at least one of grade, taste, period, and tenderness of meat included in an input meat image.

14. The system of claim 12,
wherein the user information includes a name of an owner of the user terminal, a company name, an address, an email address, and an identification, and the user information is stored in a user information table.

15. The system of claim 12,
wherein the registration meat information includes receiving a meat registration request by receiving, from the user terminal, breed, part, and a cross-sectional image of meat.

16. The system of claim 15,
wherein the registration meat information further includes a meat basic table, a fresh meat and processed meat sensory inspection table, a heated meat sensory evaluation table, an electronic tongue experiment table, and a deep aging history table,
wherein the meat basic table includes a meat management number, a meat-generating user, gender of the meat, grade, year of birth, farm, farm manager, and individual identification number,
wherein the fresh meat and processed meat sensory inspection table includes marbling, meat color, texture, and juiciness,
wherein the heated meat sensory evaluation table includes flavor, moisture, tenderness, umami, and palatability,
wherein the electronic tongue experiment table includes collagen, sourness, richness, umami, and aftertaste,
wherein the deep aging history table includes date and time, and
wherein the measuring is performed based on the registration meat information to measure at least one of taste, tenderness, and grade of the meat.

17. The system of claim 12,
wherein the memory stores code that, when executed by the processor, causes the processor to assign a unique management number to each meat and, when registration of the meat is completed, generate and provide a QR code containing completed information.

18. The system of claim 12,
wherein the memory stores code that, when executed by the processor, causes the processor to receive, from the user terminal, a request for viewing data regarding any one meat, provide data corresponding to the meat, and, when there is an error in the data, receive a data correction request from the user terminal and update the data by reflecting correction items input through the user terminal.

19. The system of claim 18,
wherein the data further includes a QR code, deep aging data, cross-sectional photographing data, sensory evaluation data, taste data, and experiment data.

20. The system of claim 13,
wherein the period includes a consumption period, a processing period, and a distribution period of the meat, and
wherein the consumption period, the processing period, and the distribution period are measured based on a second time point at which a period of the meat is measured from a first time point at which the meat is initially registered.

21. The system of claim 12,
wherein the measuring is provided based on analysis of the meat through one of an RGB camera, a spectral camera, or a hyperspectral camera.
